(19) United States
(12) Patent Application Publication
(10) Pub. No.: US 2026/0151571 A1
(43) Pub. Date: Jun. 4, 2026

(54) MEDICAMENT DELIVERY DEVICE

(71) Applicant: SHL Medical AG, Zug (CH)

(72) Inventors: John Wiklund, Nacka Strand (SE); Abhilash Joseph, Nacka Strand (SE); Stephan Müller, Zug (CH)

(21) Appl. No.: 19/123,860

(22) PCT Filed: Oct. 30, 2023
(86) PCT No.: PCT/EP2023/080260

§ 371 Date: Apr. 24, 2025

Related U.S. Application Data
(60) Provisional application No. 63/422,473, filed on Nov. 4, 2022.

(51) Int. Cl. A61M 5/32; A61M 5/19; A61M 5/20; A61M 5/315

(52) U.S. Cl. CPC A61M 5/3271; A61M 5/19; A61M 5/2033; A61M 5/31586; A61M 5/3204; A61M 2005/2013; A61M 2005/31588

(57) ABSTRACT

The present disclosure relates to a medicament delivery device comprising a housing having a proximal end and contains a container of medicament positioned within the housing in a medicament container holder. The medicament container can be dual chambered, for example a pre-filled medicament container having a needle, a dose of medicament and one or more sliding stoppers. The medicament container holder can be configured with a recess or pocket to accommodate the loading of a dual chambered container and interact with an inside surface of the housing to rotationally fix the container holder relative to the housing and the medicament container. A tubular activation member, e.g., a needle cover, is slidably positioned within a housing of the delivery device to move from a first position to a second position, and then to a final locked position. A plunger rod can only move proximally relative to the needle cover when the needle cover is in the second position.

### TECHNICAL AREA

The present disclosure concerns a medicament delivery device for administering medicament contained in a dual chambered container, more specifically an auto-injection device having a prefilled dual chambered prefilled medicament container. Specifically designed pockets or recesses in the medicament container holder facilitate loading of the dual chambered medicament container into the holder and allowing axial fixation to the housing. The delivery device has an automatic dose delivery mechanism triggered by a delivery member guard when pressed against a dose delivery site.

### BACKGROUND

A large number of medicament delivery devices on the market and developed during the last 20 years are referred to as auto-injectors because they require pushing the device against a dose delivery or injection site to activate the device such that a dose of medicament can be administered or injected directly into a patient. Once activated a compressed drive spring is released that causes a plunger rod to expel the medicament out of the delivery member associated with a container of medicament. One such well known auto-injector is disclosed in U.S. Pat. No. 9,199,038. User comfort and ease of use of auto-injectors, as well as safety features, are areas where design changes are possible that improve the overall user experience and operational performance of such devices. Likewise, the ability to deliver medicament from a multi-chambered container is another area that needs improvement.

Although medicament delivery devices such as auto-injectors often include a medicament container holder to support the medicament container 28 within the auto-injector, it can be difficult to insert the a dual chambered medicament container 28 into the medicament container holder, and there can be a risk of damaging the medicament container holder during insertion of the medicament container 28 into the medicament container holder, particularly for medicament container 28 that include bypasses. Applicant has appreciated that improvements could be made to the medicament container holder to alleviate this issue. Specifically, the present disclosure is directed to improved structural components for accepting and securely holding multi-chambered containers of medicaments in an auto-injector, improving the device activation experience, providing a smooth lockout feature to prevent user access to the used delivery member, and improving the robustness of the injection device as a whole.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site, also referred to herein as an injection site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site. Delivery member and injection needle are both used herein to refer to the component placed at the dose delivery site and that expels the medicament.

Further, the terms “longitudinal”, “longitudinally”, “axially” and “axial” refer to a direction extending from the proximal end to the distal end and along the device or components thereof, typically in the direction of the longest extension of the device and/or component. Similarly, the terms “radial” and “radially” refer to a direction extending from the axis and generally perpendicular to the longitudinal direction.

The aim of the present disclosure is to remedy or improve upon perceived drawbacks of known auto-injection devices. This aim is solved by providing a more robust design that allows a user to experience a more responsive and smoother activation of the device. Additionally, the presently disclosed designs assist in manufacturing simplicity, quality and results by providing a medicament container holder that will easily accept a multi-chamber container of medicament and will interact with the housing of the device to prevent rotation of the container holder relative to the housing. Also, the present disclosure provides a design for an easier lockout feature that prevents accidental contact with the used delivery member or an attempted reuse of the device. Preferable embodiments of the present application form the subject of the dependent claims.

According to one aspect, the medicament delivery device of the present disclosure has a tubular housing that is open at both the proximal and distal ends. An actuator having an end cap is located at the distal end and is securely fitted into the distal end opening of the housing. A plunger rod is partially assembled and positioned within a tubular proximal portion of the actuator and initially is in a first biased position. The plunger rod is hollow and has a closed proximal end. A compressed drive spring is positioned within the plunger rod and biases the closed proximal end in a proximal direction. The tubular proximal portion of the actuator has a flexible finger that releasably locks the plunger rod when it is in the first biased position. A rotator is rotationally positioned around the tubular proximal portion of the actuator such that rotation of the rotator relative to the actuator, which is rotationally and axially fixed to the housing, causes the flexible finger to unlock from the plunger rod.

Another aspect of the present disclosure comprises a medicament container holder, referred to herein also as a syringe holder, for an auto-injector, the medicament container holder comprising a tubular body extending around an axis in a circumferential direction and along the axis in an axial direction from a proximal end to a distal end, the tubular body comprising a cut-out extending from the distal end of the tubular body and a flexible expansion joint extending across the cut-out in the circumferential direction. Preferably, the expansion joint is configured to flex in the circumferential direction. The medicament container holder can help alleviate stress on the medicament container holder during and after insertion of a medicament container into the medicament container holder, particularly when the medicament container has a bypass. It can also help reduce part breakage during and after assembly. Increasing flexibility of the medicament container holder, for example by including an expansion joint, can also reduce the forces on the tools used for assembly, which can result in tools lasting longer.

Preferably, the tubular body comprises an inner surface and an outer surface, and the tubular body comprises a recess in the inner surface. Providing a recess can reduce or avoid the need for the medicament container holder to deform during insertion of a medicament container 28.

Preferably, the tubular body comprises a window, the window extending in the axial direction and spaced apart from the distal end of the tubular body and from the proximal end of the tubular body. Preferably, the tubular body comprises two windows, each window extending in the axial direction and spaced apart from the distal end of the tubular body and from the proximal end of the tubular body. Provision of a window or windows can reduce stress on the medicament container, for example by making it easier for the medicament container holder to flex during insertion of the medicament container and/or by providing space for a bypass of the medicament container to extend after the medicament container is inserted without tensioning the medicament container holder. Preferably, when two windows are provided, the windows are opposite each other relative to the axis. Provision of a window can alternatively or additionally allow the drug in the medicament container 28 to be visible after assembly of an autoinjector. Preferably, the window extends further in the axial direction than in the circumferential direction.

Preferably, the tubular body comprises a proximal end cut-out extending from the proximal end of the tubular body. This can also help increase flexibility of the medicament container holder and reduce stress on the medicament container holder and the medicament container 28, and can also help support the medicament container.

Preferably, a first of the two windows extends from the proximal end of the cut-out, and wherein a second of the two windows extends from the distal end of the cut-out. Preferably, the recess extends from the distal end of the tubular body. Preferably, the recess is deeper at the proximal end of the recess than at the distal end of the recess. Preferably, a portion of the inner surface in the recess is angled relative to the axis. These recess features can stop the medicament container from falling back out of the medicament container holder. Alternatively, the recess is deeper at the distal end of the recess than at the proximal end of the recess. This can make it easier to insert the medicament container into the medicament container holder.

Preferably, the tubular body comprises a second cut-out extending from the distal end of the tubular body, wherein the second cut-out is spaced apart from the cut-out in the circumferential direction, and wherein the proximal end of the second cut-out is adjacent to the distal end of the recess. This can further help increase medicament container holder flexibility and reduce stress on the medicament container holder. It can also make inserting the medicament container into the medicament container holder easier. Preferably, the expansion joint is closer to the distal end of the tubular body than to the proximal end of the tubular body.

Yet another aspect of the present disclosure relates to a medicament container holder having a tubular body extending around an axis in a circumferential direction and along the axis in an axial direction from a proximal end to a distal end, wherein the tubular body comprises an inner surface and an outer surface, a first cut-out in the tubular body, the first cut-out extending in the axial direction from the proximal end of the tubular body; and either a recess in the inner surface of the tubular body, the recess extending in the axial direction from the distal end of the tubular body, or a second cut-out in the tubular body, the second cut-out extending in the axial direction from the distal end of the tubular body. As with the first aspect of the present disclosure, this design can help alleviate stress on the medicament container holder during and after insertion of a medicament container 28 into the medicament container holder, particularly when the medicament container 28 has a bypass. It can also help reduce part breakage during and after assembly. Increasing flexibility of the medicament container holder, for example by including an expansion joint, can also reduce the forces on the tools used for assembly, which can result in tools lasting longer.

Preferably, the tubular body comprises a second cut-out in the tubular body, the second cut-out extending in the axial direction from the distal end of the tubular body, and wherein the tubular body comprises a recess that extends from the proximal end of the second cut-out. This can further help increase medicament container holder flexibility and reduce stress on the medicament container holder. It can also make inserting the medicament container 28 into the medicament container holder easier.

Preferably, the recess is deeper in the direction perpendicular to the axis at the distal end of the recess than at the proximal end of the recess. This can stop the medicament container from falling back out of the medicament container holder.

Preferably, a recess of the first aspect or the second aspect is configured to receive a part of a medicament container when said medicament container is inserted into the medicament container holder during assembly of a medicament delivery device. Preferably, the part of said medicament container is a bypass of said medicament container.

Another aspect of the present disclosure comprises an autoinjector comprising a medicament container holder as described above. Preferably, the autoinjector comprises a medicament container inside the medicament container holder. Preferably, the medicament container comprises a bypass. Preferably, the bypass extends in a cut-out, a recess or a window of the medicament container holder.

Yet another aspect of the present disclosure comprises a medicament container holder for an autoinjector, the medicament container holder comprising a tubular body extending around an axis in a circumferential direction and along the axis in an axial direction from a proximal end to a distal end, the tubular body comprising an inner surface and an outer surface, a window in the tubular body, the window extending in the axial direction and spaced apart from the distal end of the tubular body and from the proximal end of the tubular body, and the tubular body comprising at least one of:
a recess in the inner surface, the recess extending from the distal end of the tubular body; a cut-out extending from the distal end of the tubular body; and
an expansion joint configured to flex in the circumferential direction.

Preferably, the tubular body comprises the recess in the inner surface, and wherein the recess is deeper at the proximal end of the recess than at the distal end of the recess. Preferably, the tubular body comprises the cut-out extending from the distal end, and the tubular body comprises a recess extending in the axial direction from the proximal end of the cut-out. Preferably, the tubular body comprises a second cut-out extending from the proximal end of the tubular body.

Further, another aspect of the present disclosure comprises a medicament container holder for receiving a medicament container of an autoinjector, the medicament container holder comprising a tubular body with an outer surface and an inner surface, wherein the inner surface comprises a recess or a cut-out configured to receive a bypass portion of a medicament container, and wherein the medicament container holder comprises a C-clip at the proximal end.

The disclosed medicament deliver device has a power pack that includes a rotator having an outer surface that is arranged with guide ledges, where some of the guide ledges are extending axially in the longitudinal direction (i.e., parallel to the longitudinal axis of the delivery device) and some are inclined in relation to the longitudinal direction. The guide ledges define one or more guide tracks that accepts and provides a pathway for one or more protrusions located on a distal end of a delivery member guard, also referred to herein as a needle cover. Axial travel of the needle cover and the protrusion within the guide tracks in a distal direction results in activation of the device. The guide track can have two or more non-linear paths that reduce the force needed to activate the device such that activation occurs when the rotator has completed rotation relative to the housing and actuator, and the guide track is again linear, i.e., parallel to the longitudinal axis of the delivery device. The completed rotation of the rotator causes release of the plunger rod from the locking engagement with the free ends of flexible arms arranged on the actuator. In one possible embodiment, rotation of the rotator occurs when the protrusion on the needle cover moving axially and distally in the guide track encounters a first inclined guide ledge. As the knob moves further distally, the protrusion passes the so-called point of no return and simultaneously engages a second inclined guide ledge. Having two or more inclined guide ledges reduces the activation force that is required and experienced by the user in order to initiation the automatic injection procedure.

The outer surface of the rotator can have a radially outwardly extending protrusion positioned on the end of a flexible finger that forms part of the locking mechanism that is activated once the delivery member guard is removed from the injection site after the dose of medicament is administered by the injection device. The other part of the locking mechanism is the protrusion on the distal end of the delivery member guard. As the delivery member is moved away from the injection site, the delivery member guard will be forced out of the proximal end of the housing by a biasing element, e.g., a compression spring. The protrusion will simultaneous move proximally within a guide track on the rotator until it rides up and over a wedge-shaped protrusion at the end of the flexible finger. The design of the flexible finger must allow enough flexibility to ensure that the protrusion will not bind and can easily move past the protrusion, i.e., slide up and over, so that the protrusion will be positioned behind, i.e., on the proximal side of the wedged-shaped protrusion. When in this position any attempted movement of the delivery member guard in the distal direction will be prohibited because the protrusion will impact and be stopped by the wedged shaped protrusion.

As mentioned, the medicament delivery device has a needle cover (delivery member guard) that is slidably positioned within the proximal end of the housing. The needle cover has a radial flange located at a terminal proximal end that is design for contact and engagement with an injection site, typically a preselected location of a user's skin tissue. The radial flange functions as a bearing surface when a user pushes the outer housing proximally towards the injection site. The outward extending radial flange also provides a greater contact surface area than earlier known needle cover designs. This increased contact (surface) area provides for a better user experience and comfort, especially for those users with more than average fatty tissue. This is especially true when compared to prior known needle covers that are devoid of any radial flange feature. In a preferred design, the outer diameter of the radial flange is greater than the outer diameter of the proximal tubular portion of the needle cover and less than the outer diameter of the housing and the interior diameter of the safety cap.

To allow for a maximum possible surface area of the proximal end face of the radial flange, while still allowing the use of a protective cap that attaches to the proximal end of the delivery device, the radial flange can have one or more notches around the periphery of the radial flange. The notches can be sized to accept longitudinal ribs positioned on an inside surface of the protective or safety cap. These notches can also be configured as improved attachment points to ensure that the safety cap remains removably attached to the delivery device before its intended use.

The delivery member guard can also have a distal end designed with two parallel longitudinally extending legs. The width and lengths of the legs are configured to maximize the robustness of the delivery member guard. Preferably, the thickness (T) of legs is increased about 19% over known similar longitudinal legs. Likewise, the delivery member guard of the present disclosure has increased widths (W2, W3) of about 8% and 7%, respectively, as compared to previous designs. The distal ends of the legs are likewise configured with several features that ensure smooth functioning of the activation procedure when the delivery member guard is pushed against the injection site causing the delivery member guard to retract distally into the housing. These features can include rib guides that provide better slidability support because the rib guides are longitudinally positioned on the outside surface of each arm and form a channel configured to accept the ribs on the inside of the housing.

Another feature for enhance device operation includes housing ribs positioned longitudinally on the inside of the housing that project radially outward from the inside surface of the housing. These ribs have a projection thickness that cooperates with the rib guides on the distal end of the needle cover. The cooperation of the rib guides with the housing ribs prevents the delivery member guard from rotating relative to the housing and provides a guiding path or surface to ensure a smooth and non-binding sliding of the needle cover during activation of the device. Preferably the rib guides have a longitudinal length (L) that is approximately four times the length of similarly known rib guides. The length L is preferably proportioned to the stroke (travel) length of the needle guard during activation.

The protrusions mentioned above that are located on inner distal end surfaces of the longitudinally extending legs of the needle cover can be positioned and offset from the terminal end of the legs at a distance such that the activation of the device is either delayed or earlier as the radial flange is pressed against the injection site. Moving the protrusion in the distal direction will cause activation earlier. The protrusion can further include a chamfer or bevelled surface that is angled 20 degrees or less to prevent jamming/binding of the legs of the delivery member guard during activation as the protrusions slide in the track guide paths located on the outside surface of the rotator.

The present disclosure also can include a transport lock assembly for a powerpack of a medicament delivery device. In some circumstances the medicament delivery devices of the present disclosure can be delivered as multiple subassemblies that are connected together in a final assembly procedure. For example, in assembling an auto-injector with a loaded or compressed drive spring, one such subassembly may be a powerpack, where the compressed drive spring is biasing a plunger rod, where a rotator is configured to release the pre-tensioned plunger rod in the final assembled delivery device. For this reason, the rotator is movable, so that another element, typically part of a different sub-assembly, may interact with the rotator when the user of the medicament delivery device intends to administer a dose of medicament. During transport of the sub-assemblies, vibrations, movements and impacts may lead to accidental or unintentional release of the pre-tensioned plunger rods, which then renders the powerpacks useless for completing the device assembly. As such, it desired to ensure that the rotators are held securely or locked for shipment so that they do not accidentally activate the powerpacks during transport. At the same time, these locked powerpacks must not become difficult or complicated to unlock and/or to assemble as a result of transport locking security measures.

The present disclosure can include a transport lock assembly for the powerpack that is unlocked at the time of final assembly. One such transport lock assembly includes a powerpack having a spring-biased plunger rod, a body for holding the spring-biased plunger rod in a pre-tensioned state, a rotator for releasing the spring-biased drive member from the body, and a locking member configured to interact with the rotator, which locking member is movable, relative to the rotator, from a first state in which the rotator is immobilized, to a second state in which the rotator member is free to move, the transport lock assembly being further characterized by a housing part having a key member, which housing part is configured to receive the powerpack, and wherein assembly of the powerpack with the housing part causes the key member to move the locking member from the first state to the second state relative to the rotator.

Delaying the release of the rotator transport lock assembly is important in order to improve the injection and dose delivering functions. One design method to purposefully cause the delay is to provide a cut-out on the inside distal end of the housing of the delivery device. Such an improved design will prevent or delay rotation of the rotator during the assembly stroke so as to prevent the needle cover knob from being assembled into the wrong guide track, which in turn would make the injection device unusable. A shorter assembly stroke with an unlocked rotator would likely decreases the risk for wrong alignment.

To assist in the efficient assembly of the medicament delivery devices of the present disclosure several features can be included in the design, such as, the use of one or more visible markers than can be checked for alignment during the assembly process. For example, a marker can appear on the rotator and on an outer surface of the housing near an opening in the housing where the marker on the rotator can be viewed through the housing opening. Alignment of the two markers during the assembly process confirms the correctness of the assembly. The inclusion of chamfers and blocking gates can also assist in the assembly process. For example, including a combination of chamfers and blocking gates on the rotator outer surface can prevent misalignment of the legs of the delivery member guard when the power pack assembly, that includes the rotator, is inserted and fitted into the distal end of the housing.

A protective cap is releasably connected to the proximal end of the medicament delivery device and typically has a generally tubular body, a generally tubular medicament delivery member shield remover for removing the medicament delivery member shield. The medicament delivery member shield remover comprises an attachment element. The protective cap comprises a lid attached to the tubular body so that the medicament delivery member shield remover is held in place in this position by the end lid been in contact with the attachment element of the medicament delivery member shield remover. The attachment elements are for holding the generally tubular medicament delivery member shield remover. The lid is attached to said body for holding said medicament delivery member shield remover in a fixed position. This provides an easy and effective assembly setup of a safety cap.

Accordingly, in one possible embodiment of the present disclosure, a medicament delivery device has a housing having a proximal end and contains a pre-filled, dual chambered medicament container positioned within a container holder rotatably fixed within the housing, where the medicament container has a needle, a dose of medicament and a sliding stopper. The container holder has one or more recesses or pockets that facilitate insertion of the multi-chambered container and interact with the wall portions of windows in the housing to prevent relative rotation.

A tubular activation member, i.e., a needle cover, is slidably positioned within the housing to move from a first position to a second position, and then to a final locked position. The needle cover has two distally projecting arms and a proximal end portion terminating in a radial flange. A resilient member, e.g. a compression spring, is located in the proximal end of the needle cover, where the spring is in a compressed state when the needle cover is in the second position. A plunger rod is operatively connected to the stopper and can only move proximally relative to the needle cover when the needle cover is in the second position. Each of the distally projecting arms comprises a distal portion that forms a lock when the needle cover is in the locked position such that the needle cover is prevented from moving proximally relative to the housing and exposing the needle. The radial flange is positioned outside of the proximal end of the housing when the needle cover is in the first position, the second position, and the locked position. The radial flange has an outer diameter D3 greater than the outer diameter of the proximal end portion D2, but less than the outer diameter D1 of the proximal end of the housing.

The radial flange can have two or more notches located around the outer circumference of the radial flange, where the notches are configured to accept longitudinal ribs on the inside surface of the safety cap.

Another aspect concerns a rotator for a medicament delivery device, the rotator comprising: a tubular body extending from a proximal end to a distal end in an axial direction relative to a longitudinal axis; one or more ridges extending from a surface of the tubular body, the one or more ridges defining a track on the surface of the tubular body, the track extending in the axial direction from a distal end of the track to a proximal end of the track, the track comprising one pathway at the distal end of the track and two pathways at the proximal end of the track, wherein the two pathways at the proximal end of the track are separated by at least one of the one or more ridges; and wherein a first pathway of the two pathways at the proximal end of the track is bounded at the distal end of the first pathway by a portion of the one or more ridges, wherein the portion is angled relative to the longitudinal axis, and wherein a first section of the portion can be is at a different angle relative to the longitudinal axis than a second section of the portion. Optionally, the first section of the portion is angled at a larger angle relative to the longitudinal axis than the second portion. Additionally, or alternatively, the second section is closer to the distal end of the rotator than the first section. Additionally, or alternatively, the first section is attached to the second section. Additionally, or alternatively, the second section of the portion is angled at between 10 and 80 degrees relative to the longitudinal axis, or at between 20 and 70 degrees, or at between 25 and 50 degrees. Additionally, or alternatively, the first section of the portion is angled at between 20 and 75 degrees relative to the longitudinal axis, or at between 30 and 70 degrees, or at between 30 and 60 degrees. Additionally or alternatively, the first section is angled at between 5 and 45 degrees more relative to the longitudinal axis than the second portion, or at between 5 and 35 degrees, or at between 10 and 30 degrees.

These and other aspects of, and advantages with, the present disclosure will become apparent from the following detailed description of the disclosure and from the accompanying drawings. Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF DRAWINGS

In the following detailed description of the disclosure, reference will be made to the accompanying drawings, of which

FIG. 1 is an exploded view of a possible embodiment of the medicament delivery device

FIG. 2 presents alternative perspective views of housing 12 of the medicament delivery device of FIG. 1;

FIG. 3 shows a perspective view of an example medicament container holder;

FIG. 4 shows a different perspective view of the medicament container holder of FIG. 3;

FIG. 5 shows a perspective view of another example medicament container holder;

FIG. 6 shows a perspective view of another example medicament container holder;

FIG. 7 shows a perspective view of another example medicament container holder;

FIG. 8 shows a perspective view of another example medicament container holder;

FIG. 9 shows a perspective view of another example medicament container holder;

FIG. 10 shows a side view of the medicament container holder of FIG. 9.

FIG. 11 shows a perspective view of another example medicament container holder;

FIG. 12 shows a perspective view of another example medicament container holder;

FIG. 13 shows a side view of the medicament container holder of FIG. 12.

FIG. 14 shows a perspective view of another example medicament container holder;

FIG. 15 shows a different perspective view of the medicament container holder of FIG. 14;

FIG. 16 shows a perspective view of another example medicament container holder;

FIG. 17 shows a perspective view of another example medicament container holder;

FIG. 18 is a detailed view of the needle cover of the medicament delivery device of FIG. 1;

FIG. 19 is a detailed view of individual components that form the medicament delivery device of FIG. 1;

FIG. 20 is a detailed view of individual components that form the medicament delivery device of FIG. 1;

FIG. 21 is a detailed view of individual components that form the medicament delivery device of FIG. 1;

FIG. 22 is a detailed view of individual components that form one example of the power pack of the medicament delivery device of FIG. 1;

FIG. 23 is a detailed view of the rotator of the device of FIG. 1;

FIGS. 24, 25 and 26 show alternative perspective views of one example of a safety cap assembly and its individual components that can be used with the medicament delivery device of FIG. 1;

FIG. 27 shows another possible safety cap assembly and its individual components that can be used with the medicament delivery device of FIG. 1; and

FIG. 28 is detailed view of the rotator unlocking sequence.

### DETAILED DESCRIPTION

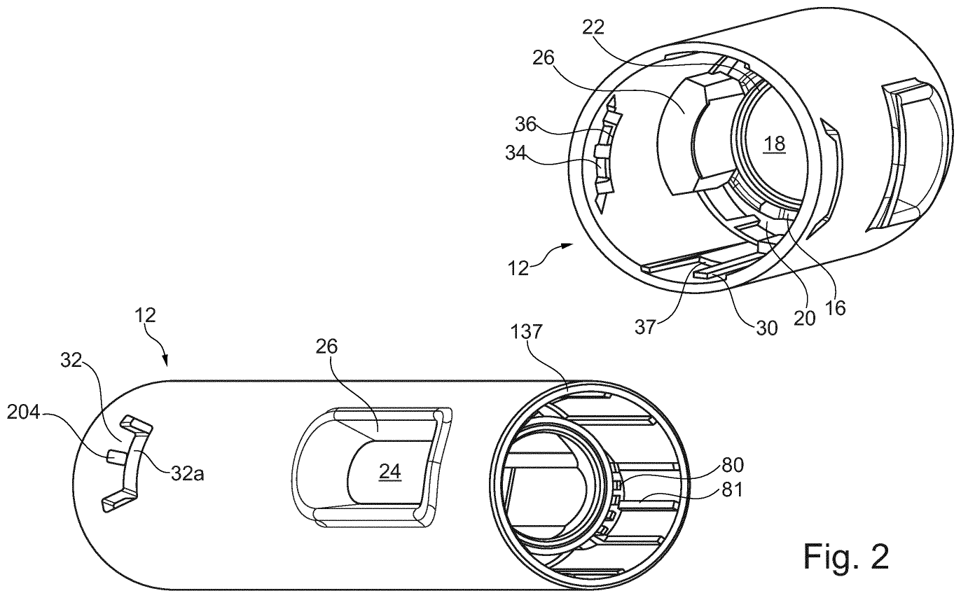

The medicament delivery device 10 shown in the drawings comprises a generally tubular housing 12 having a proximal end 14 and a distal end 15. Inside the housing a generally transversal wall 16 is arranged, FIG. 2, which wall 16 is provided with a central passage 18. Cut-outs 20 are further arranged on opposite sides of the passage 18. A seat 22 is surrounding the passage 18. Generally rectangular windows 24 are arranged in the housing, which windows 24 are arranged with inwardly directed wall sections 26. The proximal parts of the wall sections 26 are attached to or form part of the transversal wall 16. At the distal end of the housing on the outer surface one or more anti-roll features 12b can be included to prevent the medical delivery device from rolling on a flat surface, for example, preventing the delivery device from rolling off a table top and landing on the ground.

A number of longitudinally extending ribs 30 and 80 are arranged on the inner surface of the housing at both the proximal and distal ends of the housing 12. An inwardly directed protrusion 32 positioned as part of a cut-out 32a at the distal end of the housing has generally radially flexing tongues 34 that are arranged with inwardly extending ledges 36 at their free ends. An assembly alignment indicator 204 can be located on the outer surface of the proximal end of the housing 12 and is positioned near the protrusion 32 and associated cut-out portion 32a of the housing.

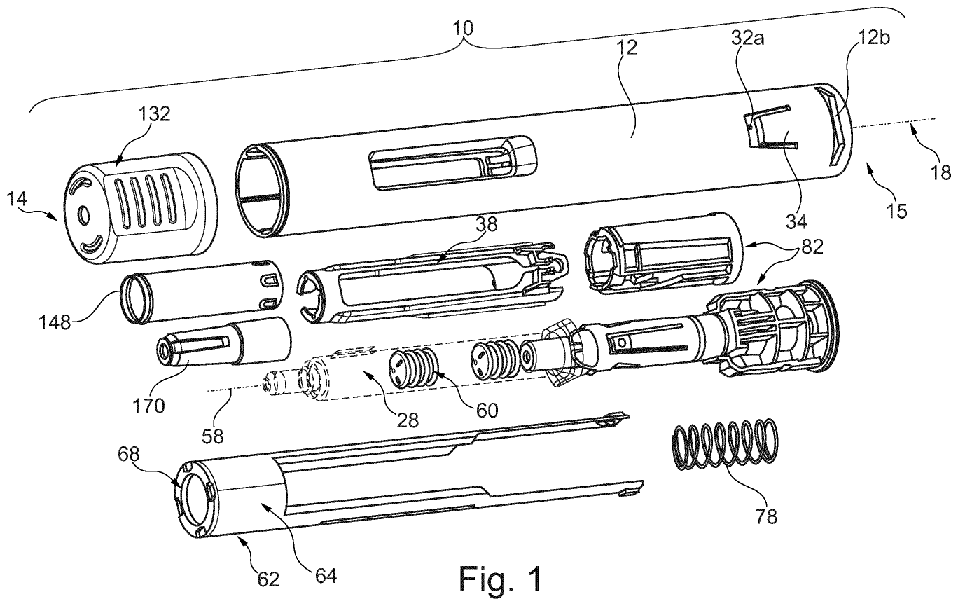

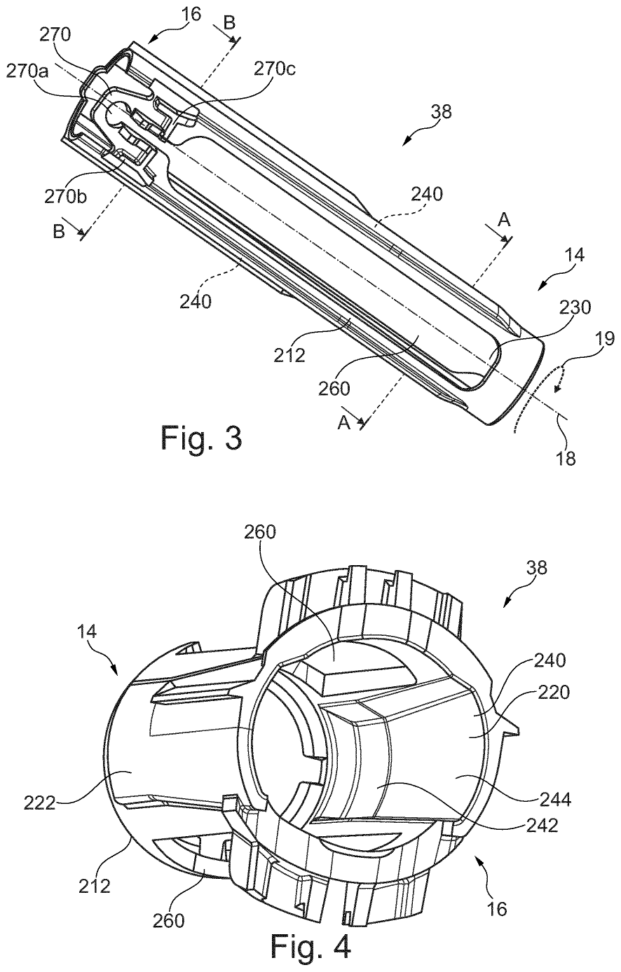

Inside the housing 12, a medicament container holder 38 is arranged coaxially. FIG. 3 shows one possible embodiment of a medicament container holder 38 for the medicament delivery device 10 illustrated in FIG. 1, which can be configured as an auto-injector. In the illustrated embodiment, the medicament container holder 38 comprises a tubular body 212 extending in an axial direction relative to an axis 18 from a proximal end 14 to a distal end 16, and in a circumferential direction 19 around the axis 18. Located at the distal end 16 are alignment and locking tabs 270b, 270c. Where the alignment tab 270b cooperates with the inside of the housing 12 to axially guide the medicament container holder 38 in the proximal direction when it is inserted into the open distal end of the housing 12, such as during the manufacture of the medicament delivery device 10. As the medicament container holder 38 is pushed further into the housing 12, tab 270c will eventually cooperate with cut-out 32a to form a snap fit with the housing 12 defining a transport lock that prevents relative rotation of the of the medicament container holder 38 relative to the housing 12.

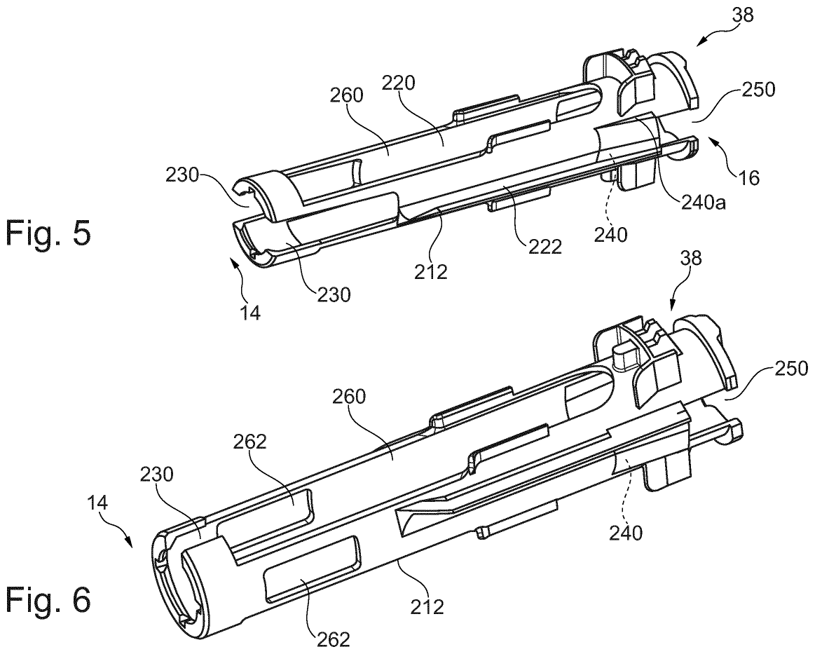

In order to help with reducing stress on the medicament container holder 38, for example, a number of possible medicament container holder structures are envisioned. In one example, the tubular body 212 comprises a cut-out 230 extending from the distal end 16 of the tubular body 212 and a flexible expansion joint 270 extending across the cut-out 230 in the circumferential direction. In another example (see FIGS. 4 and 5), the tubular body 212 comprises an inner surface 220, an outer surface 222 and a first cut-out 230, the first cut-out 230 extending in the axial direction from the proximal end 14 of the tubular body 212; and either a recess 240 in the inner surface 220 of the tubular body 12, the recess 240 extending in the axial direction from the distal end 16 of the tubular body, or a second cut-out 250 in the tubular body 212, the second cut-out 250 extending in the axial direction from the distal end 16 of the tubular body 212.

Two windows 260 can be provided and are arranged opposite one another relative to the axis 18. Two recesses 240 can also be provided, and are arranged opposite each other relative to the axis 18 (a recess 240 can be seen directly in FIG. 2, for example; in some Figures such as FIG. 1, where the recess is not directly visible, the structure that forms the recess is marked with reference numeral 240 but with a dotted line). The windows 260 and the recesses 240 overlap each other in the axial direction and are spaced apart in the circumferential direction 19 (although they could alternatively be adjacent to one another or even overlapping in the circumferential direction). In other words, when going in the circumferential direction around the tubular body 212, there is a first window 260, then a first recess 240, then a second window 260, then a second recess 240, and then the first window 260 again.

A cut-out 230 extending in the axial direction from the proximal end 14 of the tubular body 212 is also provided. The distal end of the cut-out 230 is adjacent to the proximal end of the second of the two windows 260 in this example. In this example, the proximal end of the tubular body 212 therefore extends around the axis 19 with the exception of the first cut-out 230. The provision of a cut-out 230 makes the shape of the proximal end of the tubular body 212 into a feature that can be described as a C-clip. This can support a medicament container 28 in the medicament container holder 38 by supporting the proximal end of the

Two recesses 240 can be provided, with the two recesses 240 being the same as one another. The recesses 240 are deeper at the proximal end than the distal end. The recesses 240 are also narrower in the circumferential direction at the proximal end than at the distal end. The recesses 240 can be thought of as two parts, a proximal portion 242 and a distal portion 244. The surface of the distal portion 244 slopes in the axial direction relative to the axis 19, and the distal portion 244 of the recess 240 is therefore deeper at the proximal end of the distal portion 244 than at the distal end of the distal portion 244. The width of the distal portion 244 in the circumferential direction also reduces in the axial direction, with the distal portion 244 being wider in the circumferential direction at the distal end of the distal portion 244 than at the proximal end of the distal portion 244. Practically speaking, being wider at the distal end of the distal portion can make it easier to align a bypass of a medicament container 28 with the recess. Being deeper at the proximal portion can give space for the bypass of the medicament container 28 once the medicament container 28 is fully inserted in the medicament container holder 38 and can also make it harder for the medicament container 28 to simply fall out, due to the shallower parts of the recess at the distal end. This will be described in more detail below.

In the example embodiment FIGS. 1 and 2, the medicament container holder 38 extends fully around the axis 19 at the distal end 16 apart from a third cut-out 270a in which an expansion joint 270 is provided. The expansion joint 270 is provided at the distal end of the first of the two windows 260. The expansion joint 270 is flexible, and deformation of the expansion joint 270 can allow the diameter of the distal end to increase when a medicament container 28 is being added into the medicament container holder 38. It can also help remove (eject or de-mould) the component during injection moulding, as the flexibility of the expansion joint allows for easier removal of medicament container holders with an under-cut from the core pin, for example an under-cut (overhang) due to a recess that is deeper at the proximal end. In this particular example, the expansion joint 270 is V-shaped, but other shapes are also possible. In general, the expansion joint is optional, as outlined in more detail below.

One of the windows 260 extends from the first cut-out 230 (and can be considered to be a part of the cut-out 230), although the window 260 could also be spaced apart from the first cut-out in the axial direction. The other window 260 is adjacent to the expansion joint 270 in the longitudinal direction. Although this window 260 could alternatively be spaced apart from the expansion joint 270, placing the window 260 adjacent to the expansion joint 270 in the axial direction can make it easier for the medicament container holder 38 to flex to accommodate a medicament container 28. In other words, the third cut-out extends from the distal end and the expansion joint 270 is in the third cut-out, with the window being an optional extension of the third cut-out.

Instead of an expansion joint, a cut-out (a distal end cut-out) 250 (see FIGS. 5 &6) at the position of the expansion joint could instead be provided (without an expansion joint in the cut-out). This would also allow for expansion of the diameter of the distal end of the medicament container holder 38 as a medicament container 28 is pushed into the medicament container holder 38. In some cases an expansion joint would be preferable, for example to provide structural integrity for the medicament container holder 38, for example. In some cases, however, a distal end cut-out would be preferable; for example, just providing a cut-out could simplify medicament container holder manufacture.

When a medicament container 28 with a bypass or bypasses is being inserted into a medicament container holder 38 as described herein, the proximal end of the medicament container 28 (typically including a medicament delivery member such as a needle) is inserted into the distal end of the medicament container holder 38. In the example embodiment of FIGS. 1 and 2, the bypasses are aligned with the recesses. As the bypasses enter the recesses, the recesses can move apart from one another slightly to accommodate the bypasses, because the expansion joint is flexible. In this example, the recesses are deeper at the proximal end of the recesses than at the distal end of the recesses such that once the bypasses reach the proximal end they have more space and no longer need to deform the medicament container holder 38, which can return to its original non-tensioned state.

In the example of FIGS. 1 and 2, the recess depth is greater at the proximal end of the recess than at the distal end of the recess, with a sloped recess surface (inner surface of the tubular body), which means that there is more space towards the proximal end to accommodate the bypass or bypasses. As a result, as the medicament container 28 is pushed deeper into the medicament container holder 38, there is less and less need for the medicament container holder 38 to flex to accommodate the bypass.

In addition to the expansion joint (or alternatively to the expansion joint, in examples without expansion joints), the rest of the tubular body can also flex, and several design features of the medicament container holders described herein assist with this flexibility. For example, the proximal end can flex and increase its diameter slightly due to the proximal end cut-out 230 (see FIGS. 5&6). The provision of windows 260 can also allow for flexing, particularly of the central portion of medicament container holder 38. Finally, there is a certain amount of flex available within the structure even at points where there is no cut-out or window (with the structure of the medicament container holder 38 becoming slightly oval to accommodate the bypass or bypasses), although this can be a source of considerable stress for the medicament container holder 38 and this deformation, whilst still possible, is typically minimised or avoided completely by the structure of the example medicament container holders described herein. The examples herein have windows, cut-outs and/or recesses for most or all of their length in the axial direction or for the entirety of their length in the axial direction—this combination helps with reducing stresses during insertion of a medicament container 28 with a bypass or bypasses.

FIG. 5 shows another example medicament container holder 38 of the present disclosure. Two proximal end cut-outs 230 extend from the proximal end 14 in the axial direction. Two distal end cut-outs 250 extend from the distal end 16 in the axial direction. At the proximal end of each of the two distal end cut-outs 250, a recess 240 extends in the axial direction. These recesses 240 can be considered as entry chamfers, with the recess 240 being sloped and the distal end of the recess 240 being deeper than the proximal end of the recess 240. The proximal end cut-outs 230 are aligned in the axial direction with the distal end cut-outs 250 (that is, they overlap in the circumferential direction). Two windows 260 are also provided; the windows 260 are spaced apart from the cut-outs 230, 250 in the circumferential direction (although in examples where the cut-outs 230, 250 do not overlap the windows 260 in the axial direction, the windows 260 can be adjacent to or overlap with the cut-outs 230, 250 in the circumferential direction). When a medicament container 28 with two bypasses is placed in the medicament container holder 38, the bypasses would extend in the proximal end cut-outs. In general, provision of a cut-out (or a recess as in FIG. 1) to accommodate a bypass after a medicament container 28 with a bypass has been inserted into the medicament container holder 38 is beneficial, as it can reduce or avoid ongoing distortion of the medicament container holder 38 by the bypass during subsequent transport and storage. Having two proximal end cut-outs means that the medicament container holder 38 has 180-degree rotational symmetry, which can make orientation during assembly easier.

FIG. 6 shows yet another example medicament container holder 38 of the present disclosure. Instead of having two proximal end cut-outs extending from the proximal end 14 as in the example in FIG. 5, two windows 262 spaced apart from the proximal end 14 are instead provided, and a single proximal end cut-out 230 is provided at the proximal end 14 (providing a C-clip structure at the proximal end of the tubular body). The structure at the distal end is the same as described for FIG. 5. When a medicament container 28 with two bypasses is placed in the medicament container holder 38, the bypasses would extend in the window or cut-outs that are spaced apart from the proximal end 14.

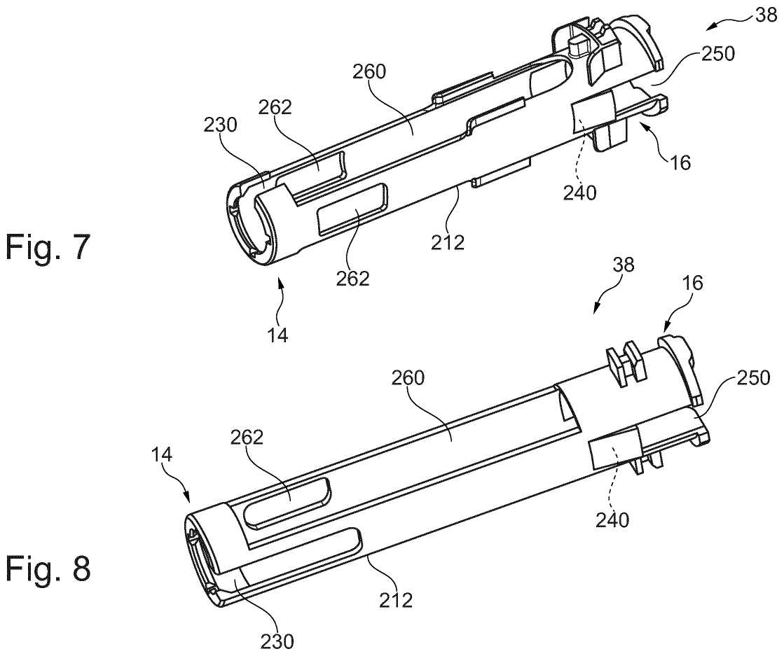

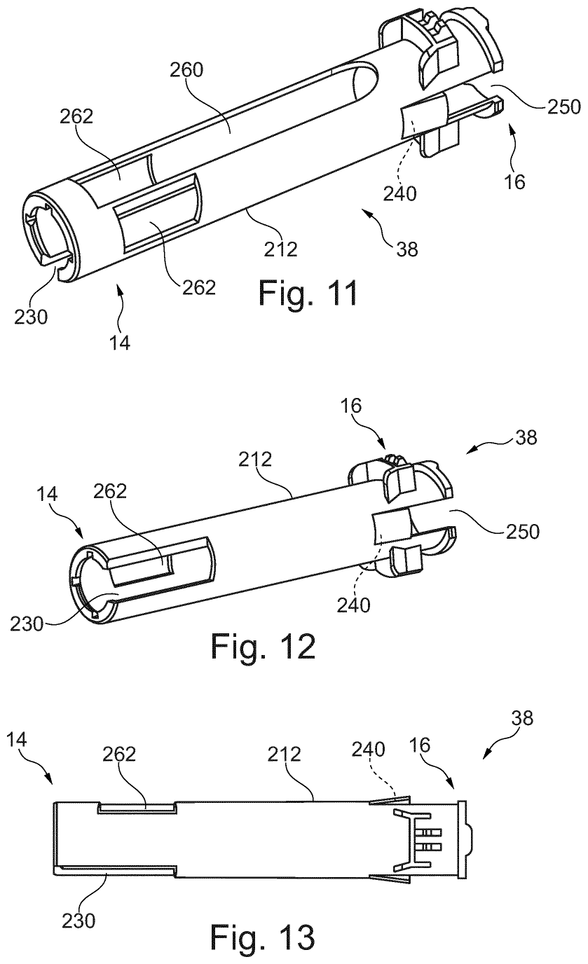

FIG. 7 shows another medicament container holder 38 of the present disclosure that is very similar to the example in FIG. 6, as does FIG. 11. FIG. 8 shows a medicament container holder 38 with an intermediate design in between the designs shown in FIG. 5 and in FIGS. 6 and 7. The design can basically be described as being the same as that in FIGS. 6 and 7, but with the proximal end cut-out rotated 90 degrees in the circumferential direction so that one of the windows 262 is aligned in the axial direction with the proximal end cut-out (rather than being aligned in the axial direction with the window 260).

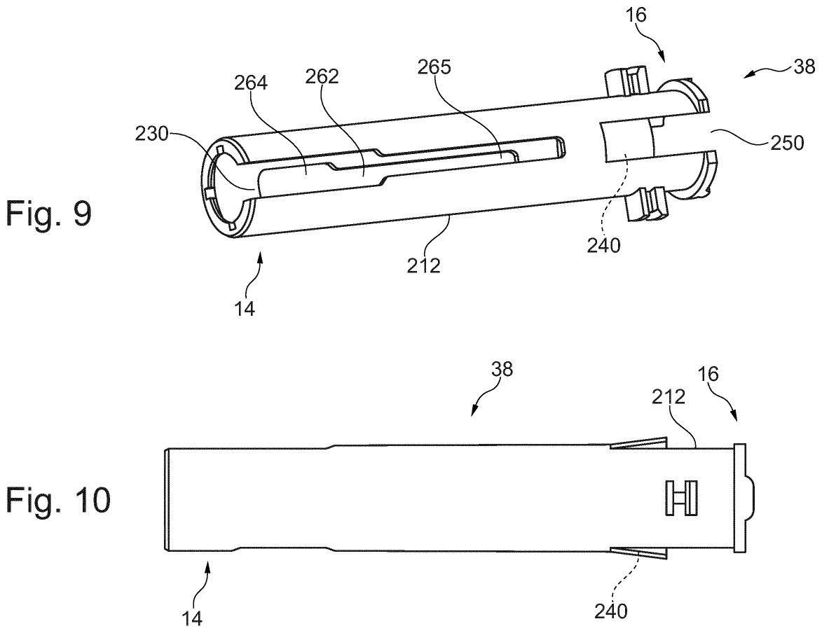

FIGS. 9 and 10 show yet another example of a medicament container holder 38 of the present disclosure. The design at the distal end is the same as described in the above-mentioned holders 38, such as FIG. 5. The medicament container holder 38 of FIGS. 9 and 10 has a single proximal end cut-out 230. There are two windows 262, with one window 262 aligned in the axial direction with the proximal end cut-out 230 and the other window 262 on the other side of the axis relative to the proximal end cut-out 230. The distal end cut-outs 250 are aligned in the axial direction with the windows 262. The two windows 262 have a proximal portion 264 and a distal portion 265, with the proximal portion 264 being wider in the circumferential direction than the distal portion 265. When a medicament container 28 is placed in the medicament container holder 38, the bypasses are inside the proximal portion 264 of the window 262. The thinner distal portion 265 of the window 262 can also partly or entirely receive the bypasses during insertion of the medicament container 28 into the medicament container holder 38, which can reduce stress on the medicament container holder 38 during insertion of the medicament container 28.

FIGS. 12 and 13 show another example medicament container holder 38 where at the distal end 16 is the same as previously described. At the proximal end 14, a single proximal end cut-out 230 is provided (resulting again in a C-clip design) which is aligned with one of the windows 262. Two windows 262 are provided, with the windows 262 aligned in the axial direction with the distal end cut-outs 250.

The designs in FIGS. 9 and 10 and in FIGS. 12 and 13 do not include other windows 260. In general, the other windows 260 in some of the other embodiments are optional, although they can provide several benefits when provided, including reduction of stress/strain on the medicament container holder 38 during insertion of the medicament container 28 and allowing visibility of the drugs within the medicament container 28 after the medicament container 28 has been placed in the medicament container holder 38. In designs without a window 260 that allows for visibility of the drug, part or all of the medicament container holder 38 can be made transparent. This can allow for visibility of the drug despite the lack of a bespoke drug viewing window. Windows 262, which are typically provided to allow space for the bypasses of the medicament container 28 once the medicament container 28 is placed in the medicament container holder 38, would not necessarily allow clear viewing of the drug due to interference from the bypasses.

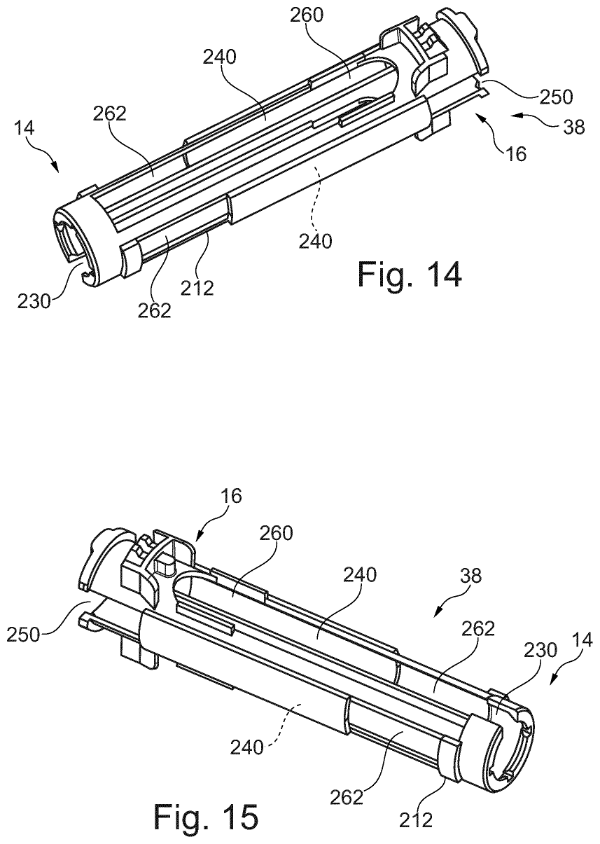

FIGS. 14 and 15 show another example medicament container holder 38, where at the distal end of the tubular body 212, two distal end cut-outs 250 are provided opposite one another relative to the axis. At the proximal end of each distal end cut-out 250, a recess extends in the axial direction. At the proximal end of each recess, a window 262 extends in the axial direction. In between the recesses, two windows 260 are provided. At the proximal end of the tubular body 212, a single proximal end cut-out is provided (i.e. a C-clip design), with the proximal end cut-out aligned with one of the windows 60 in the axial direction.

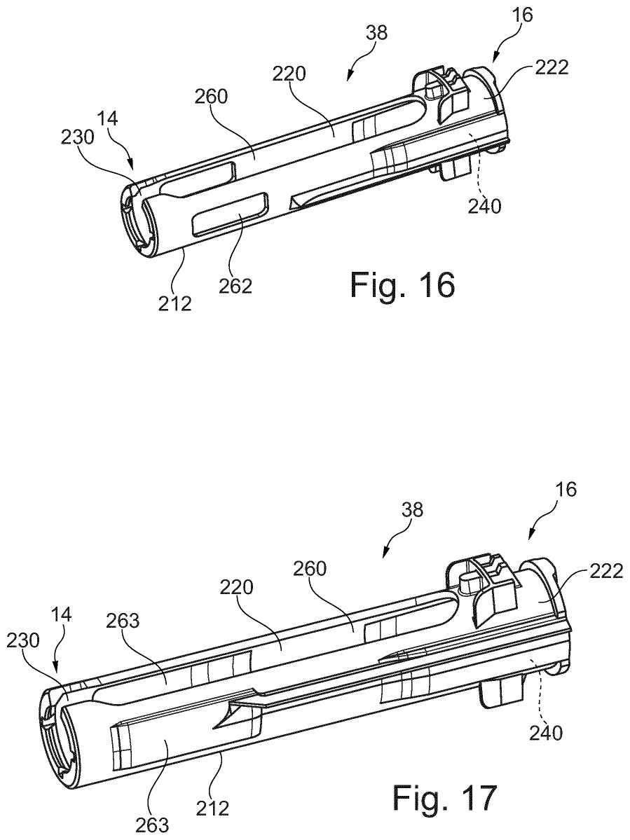

FIG. 16 shows a further example of the medicament container holder 38 of the present disclosure that is similar to the holders illustrated in FIGS. 1 and 6 in particular. Two recesses 240 extend from the distal end of the tubular body 212. There are no distal end cut-outs. The proximal end has a C-clip; that is, only one proximal end cut-out 230. A window 260 extends from the distal end of the proximal end cut-out 230, and a second window (not visible) is also provided opposite the window relative to the axis. Two windows 262 are also provided. FIG. 17 shows another example medicament container holder 38. This medicament container holder 38 is the same as the example in FIG. 16, but instead comprises two bypass recesses 263 instead of the windows 262.

In general, the medicament container holder 38 is a medicament container holder 38 for a medicament delivery device 10 such as an autoinjector. Although the medicament container holders described herein are useful for medicament containers 28 in general, they can be particularly beneficial when the medicament container 28, e.g. syringe, cartridge or ampoule, that is inserted into the medicament container holder 38 is a dual-chamber medicament container with at least one bypass (external bypass), as the bypass tends to increase the width of the medicament container 28 by protruding out further from its central axis compared to the rest of the container 28. In the context of this application, a medicament container 28 typically is a medicament container 28 pre-filled with medicament that can be included within a medicament delivery device 10 such as an autoinjector. The medicament container 28 typically includes a medicament delivery member such as a needle or cannula 58, along with a needle shield 170, 171 and shield remover 148 as described above and illustrated in FIG. 1. The medicament container 28 can contain one or more stoppers 60 of resilient material that is slidably movable inside the tubular body of the medicament container 28. In most embodiments, the medicament container 28 is a syringe and the medicament container holder 38 may be best described as a syringe holder.

The medicament container 28, preferably a pre-filled medicament container 28, could typically have two bypasses at opposite sides of the medicament container 28, or could have one, three or more bypasses spaced around the medicament container 28 in the circumferential direction, and the medicament container holder 38 may be designed accordingly. As such, although two recesses are shown in the examples, in each case only one recess is required (and the same for windows and cut-outs in the examples). In some examples, two recesses or cut-outs are provided opposite each other relative to the axis even though there is only one bypass, as this can make assembly easier by providing 180-degree symmetry (though some features such as the C-clip do not need 180-degree symmetry), allowing the syringe holder 38 to function in two different orientations even with a single bypass.

The tubular body 212 in the various embodiments herein is shown as a cylindrical body with a circular cross section perpendicular to the axis, but other shapes are also possible. The inner surface 220 is the surface of the tubular body facing generally towards the axis, and the outer surface 222 is the surface of the tubular body facing generally away from the axis. The cut-out 230 (proximal end cut-out) is shown on the opposite side of the axis relative to the expansion joint 270 in FIG. 3, but could be at a different location, for example on the same side (overlapping in the circumferential direction) or 90 degrees removed in the circumferential direction (rather than the 180 degrees shown in FIG. 3). Rather than one cut-out 230, two or more cut-outs could be provided. With one proximal end cut-out, the proximal end of the syringe holder 38 can be thought of as a C-clip. Alternatively, some examples can include no proximal end cut-outs 230.

Similarly, where recesses 240 are provided, the examples herein show two recesses. However, one, three or more recesses could alternatively be provided, and the examples shown herein could all be modified to only have one recess instead of the two recesses shown. A recess such as the recess in FIG. 3 can be replaced by a cut-out, or combined with a cut-out (as in the examples in FIG. 5 onwards). In general, when a recess is provided, a portion of the inner surface of the tubular body is the surface of the recess. The surface of the recess could be parallel to the axis (e.g. FIG. 15), or partially parallel to the axis (proximal portion 242 in FIG. 4) and partially angled relative to the axis (distal portion 240 in FIG. 4). Where part or all of the axis is angled relative to the axis, it can be angled so that the surface is angled to face towards the distal end (e.g. FIGS. 5 to 13 and 16 to 17) or angled to face towards the proximal end (e.g. FIG. 4). A particular shape of recess is shown in FIG. 3, with a proximal portion 242 and a distal portion 244, but other shapes of recess could also be used in the example in FIG. 3, for example a recess shaped as in FIG. 5 or FIG. 14.

Typically, the distal end cut-out 250/recess 240 is aligned with the proximal end cut-out 230/the window 262/the bypass recess 263 for the bypass, which can help with assembly. As mentioned above, distal end cut-outs 250 can have expansion joints 270 in or can be without expansion joints 270. In general, one or more distal end cut-outs can be provided, with none, some, or all of the distal end cut-outs having expansion joints in. This description describes various windows (cut-outs). The description distinguishes between windows 260 (which typically provide a window through which the drug could be seen) and windows 262 (which typically provide a space for the bypass of the medicament container 28), though the two can be combined or interchanged and are not necessarily structurally different. In general, windows 262 can also be replaced with bypass recesses such as those in FIG. 17. In general, there are several different possible functions for windows described herein, including to allow visibility of the drug (e.g. FIG. 3), to allow space for a bypass of the medicament container 28 during or after assembly (e.g. FIG. 9), and to allow or help allow flexing of the medicament container holder 38 when the medicament container 28 is inserted. Any given window can have one or more functions, depending on the particular design of the medicament container holder 38.

In general, windows 260 are optional and could be removed from the examples herein, although when provided they can also help reduce stress on the medicament container holder 38 during assembly, for example by making it easier for the medicament container holder 38 to flex to accommodate bypasses. One example of this is in FIG. 5, where the provision of a window or windows 260 can help allow the medicament container holder 38 to flex and allow a medicament container 28 to pass into the medicament container holder 38.

Various different shapes for windows are possible beyond those shown in the examples, and would depend on the particular device in which the medicament container holder 38 is being used and on the shape of the medicament container 28 in which the medicament container holder 38 is being used. Windows are typically rectangular or oval with the longer side extending in the axial direction, and may have right-angled corners (e.g. window 260 in FIG. 8), slightly curved corners (e.g. window 262 in FIG. 8), or broadly curved corners (e.g. distal end of window 260 in FIG. 11). Irregularly shaped windows are also possible, such as window 62 in FIG. 9.

Similarly, the shapes of the cut-outs described herein could be varied, with rectangular shaped cut-outs with right-angled corners, slightly curved corners, broadly curved corners or a mix of corner shapes being possible. Irregularly shaped cut-outs are also an option.

Although an expansion joint 270 is only shown in the example in FIG. 3, an expansion joint could also be provided in the other examples described herein. More than one expansion joint could also be provided; for example, a second expansion joint could be provided opposite the first expansion joint (i.e. on the other side of the axis) in the example shown in FIG. 3.

One possible expansion joint shape is shown in FIG. 3, namely a V-shaped expansion joint. The protrusions extending radially away from the expansion joint are not necessary for the functioning of the expansion joint. Other shapes of expansion joints are possible, for example W-shaped, S-shaped or spring-shaped expansion joints. The expansion joint would typically flex in the circumferential direction, as this allows for the expansion joint to take up a minimum of space in the radial direction, but flexing in the radial direction instead or as well is also an option.

A number of the cut-outs and windows are adjacent to one another, meaning that often a cut-out and its adjacent window effectively provide a single larger cut-out. This is the case, for example, in FIGS. 6, 7, 11 and 15 (cut-out 230 and window 260), and in FIGS. 9 and 12 (cut-out 230 and window 262). It is also effectively the case in FIGS. 5 and 8, for example, where the distal end of the cut-out(s) 230 could be considered to be a window 262.

Another benefit to having a pocket or recess incorporated into the design of the medicament container holder 38 is to assist in positioning of the container holder 38 relative to the housing 12. The recess 240 can be located at an axial position along the container holder 38 such that when the container holder 38 is inserted into housing 12 during assembly of the medicament delivery device 10, the recess will be positioned within window 24. Since window 24 is arranged with inwardly directed wall section 26 the protruding edge 240a of recess 240 (see FIG. 5) will abut the wall section 26. This abutment will prevent relative rotation between the holder 38 and housing 12 because the wall section 26 will act as a hard stop if the holder 38 is subjected to a rotational force.

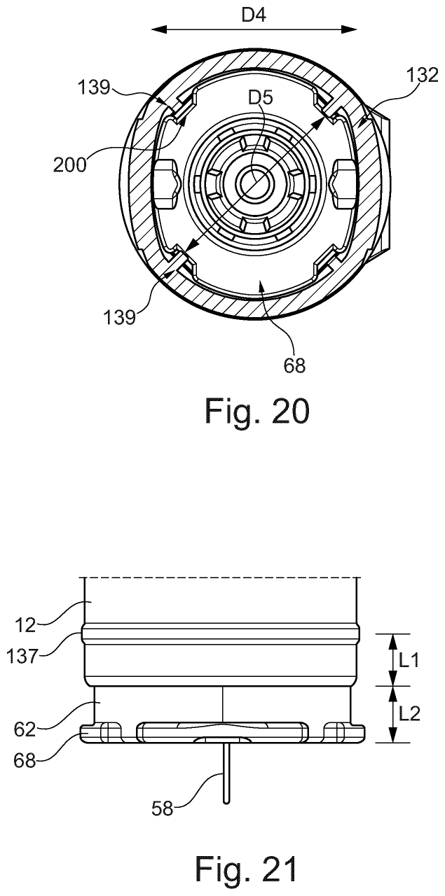

The medicament delivery device 10 further comprises a medicament delivery member guard 62, also referred to as a needle cover (see FIGS. 1 & 18). The delivery member guard 62 comprises a proximal generally tubular body 64 provided with a central passage 66 of a diameter to allow passage of needle 58 during medicament delivery. The needle guard 62 terminates at the proximal end in a transversal radial flange 68 that defines an injection site bearing surface. The terminal proximal end of the needle guard 62 can contain a radial flange 68 that can have an outside diameter D3 (see FIG. 19) that is greater than the outside diameter D2 of the proximal end 64 but is less than the outer diameter of D1 of housing 12. Two or more notches 200 are located around the circumference of radial flange 68 and are sized to accept longitudinal ribs 139 located on the inside surface of safety cap 132 (see FIG. 20). The ribs 139 define a diameter D5 that is less than the inside diameter D4 of cap 132, which is greater than the outside diameter D3 of radial flange 68. Engagement of ribs 139 and notches 200 prevents relative rotation of the cap 132 with respect to the housing 12 and needle cover 62. Cap 132 connects with housing 12 through rim 137 (see FIG. 21).

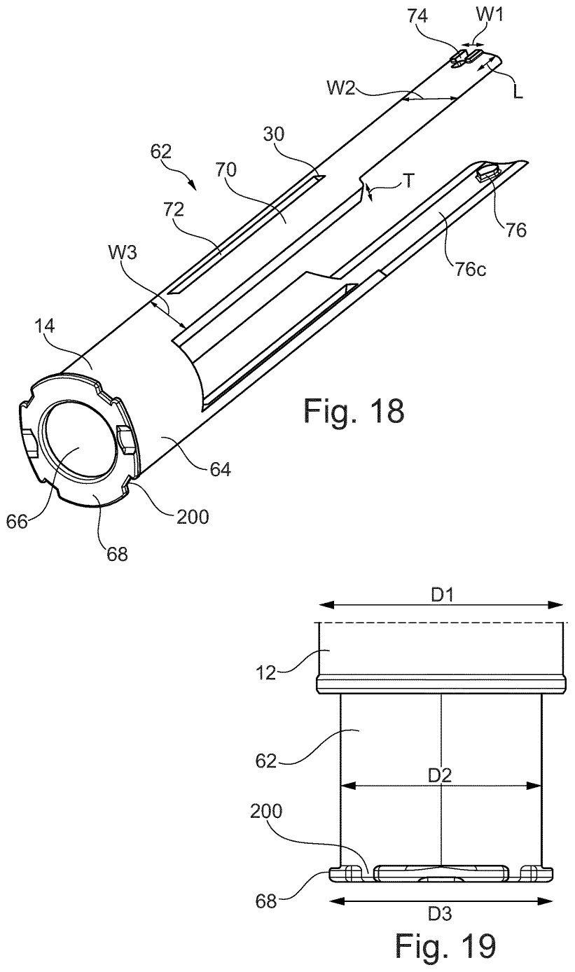

Two oppositely positioned arms 70 are arranged to the distal area of the body 64 and extending in the distal direction. The arms 70 are arranged with longitudinal slits 72 which are to cooperate with the longitudinal ribs 30 of the interior of the housing (see FIG. 18). The arms 70 are characterized by two different widths, namely W2 and W3, where W2 is less than W3, and both widths are selected to provide sufficient rigidity to prevent binding or bending of the needle guard 62 as the radial flange 68 is pushed against an injection site prior to completing activation of the medicament delivery device 10 and commencing dose delivery. At the distal end of the arms 70, outwardly radial directed ledges 74 are provided. Preferably, two such ledges are used on each distal end of the arms 70 and are oriented and spaced longitudinally to define a channel having a width W1. This channel is sized accept longitudinal rib 37 on the inside of housing 12 (see FIG. 2) such that rib 37 functions as a guide track for the distal sliding movement of the needle guard 62 relative to the housing 12 as the medicament delivery device 10 is pressed and pushed against an injection site.

On the inner surface of the arms 70 there are radially inward directed protrusions 76 (see FIG. 18). The protrusions 76 are preferably tear dropped shape and have a bevelled or chamfered edge less than 58 degrees, preferably 20 degrees or less, and most preferably 15 or 8 degrees, as measured relative to the inner surface 76c of the leg 70. These angled chamfers prevent or minimize an undesirable failure mode of delayed activation. The distance of the protrusions is predetermined to achieve a desired needle injection depth. The protrusions 76 are positioned a distance from the terminal distal end of legs 70. As indicated, the delivery member guard 62 can also have a distal end designed with two parallel longitudinally extending legs. The width and lengths of the legs are configured to maximize the robustness of the delivery member guard 62. Preferably, the thickness (T) of legs is increased as much as possible, preferably about 19% over known similar longitudinal legs. Likewise, the delivery member guard 62 of the present disclosure has increased widths (W2, W3) of about 8% and 7%, respectively, over known devices using similar needle guards.

A medicament delivery member guard spring 78 is further arranged between a distally directed surface of the radial flange 68 of the needle cover 62 and a proximally directed surface of a wall on an inside of the proximal end 14 of the housing 12. In this regard, proximally directed support protrusions 80 (see FIG. 2) are provided on the wall for supporting the medicament delivery member guard spring 78 and preventing it from accidentally interacting with the arms 70 of the needle cover 62. The spring 78 biases the needle cover 62 before, during and after activation of the medicament delivery device 10 and completion of the medicament medicant delivery at the injection site. FIG. 21 illustrates the spatial relationships of the housing 12, needle cover 62 and proximal end of the delivery member 58 (which in the illustrated embodiment is a needle) when the radial flange 68 has been pressed against an injection site and medicament delivery has begun. The housing 12 and/or needle cover 62 lengths are preselected and designed such that L2 is 1.5 mm or greater so as to avoid contact of the terminal proximal end of the housing 12 with the injection site, which can cause discomfort to the user. Likewise, rim 137 is located a distance L1 from the terminal proximal end of the housing to avoid interaction with the injection site. L1 is preferably 3 mm or greater.

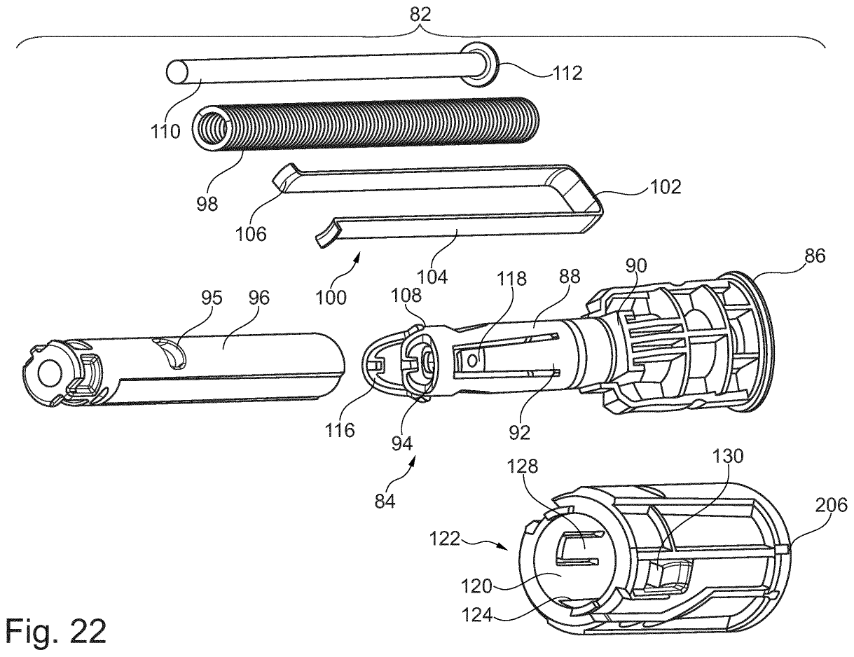

The medicament delivery device 10 shown in FIG. 1 also comprises a power pack or drive unit 82. FIG. 22 shows the details of the power pack 82 that includes an actuator 84 provided with a distal portion forming an end cap 86 of the distal end of the housing 12 of the medicament delivery device 10 when the power pack 82 is inserted and fitted into the housing 12. The proximal part of the actuator 84 comprising a generally elongated tubular body 88. A transversal support surface 90 is arranged in the area between the end cap 86 and the body 88, which support surface 90 is designed to cooperate with the ledges 36 of the tongues 34 on the housing 12 for locking the actuator 84, and thus the power pack, to the housing 12 when the medicament delivery device 10 is fully assembled. The body 88 is further arranged with proximally directed arms 92 that are flexible in a generally radial direction. The free ends of the arms 92 are provided with inwardly directed protrusions 94. These inwardly directed protrusions 94 are arranged to fit into and cooperate with recesses or cut-outs 95 in an elongated plunger rod 96, which plunger rod 96 is a hollow tubular structure having a closed proximal end and an open distal end, where the plunger rod 96 is intended to fit into and be coaxial with the body 88 of the actuator 84.

The rotator 122 also can have an alignment indicator that can be visible through cut-out 32a at the distal end of the housing 12. During assembly of the power pack 82 into the housing 12, the rotator 122 is positioned and oriented with respect to the housing 12 such that alignment indicator 204 is aligned with alignment indicator 206 on the rotator 122. Alignment indicator 206 could be a small, but detectable, sink-notch, that is visible through cut-out 32a after final assembly and used to verify rotator 122 positioning throughout the life cycle of the medicament delivery device 10.

Further, a drive spring 98 is arranged partially inside the hollow portion 96a of the plunger rod 96 and a guide rod 110 is arranged inside the drive spring 98. The guide rod 110 is provided with a disk 112 at its distal end. A U-shaped bracket 100 has a transversal distal part 102 and two proximally extending arms 104 on either side of, and outside, the drive spring 98. The ends of the arms 104 are arranged with outwardly extending ledges 106, which ledges 106 are to be in contact with proximally directed edge surfaces 108 of the body 88 of the actuator 84. The drive spring 98 is thus arranged between a proximal end wall 114 of the plunger rod 96 and the transversal distal part 102 of the bracket 100 via the disk 112 of the guide rod 110. Further, at the proximal end of the body 88 there are arc-shaped support elements 116 that project proximally and are flexible in the generally longitudinal direction. These elements 116 are intended to be in contact with and support the medicament container 28 so as to minimize relative axial movement of the container 28 relative to the medicament container holder 38. Preferably, the elements 116 exert a biasing force in the proximal direction to prevent unwanted axial movement of the medicament container 28. In other words, the contact or engagement by elements 116 can impart a biasing force in the proximal direction on the medicament container holder 38 and/or the medicament container 28 itself.

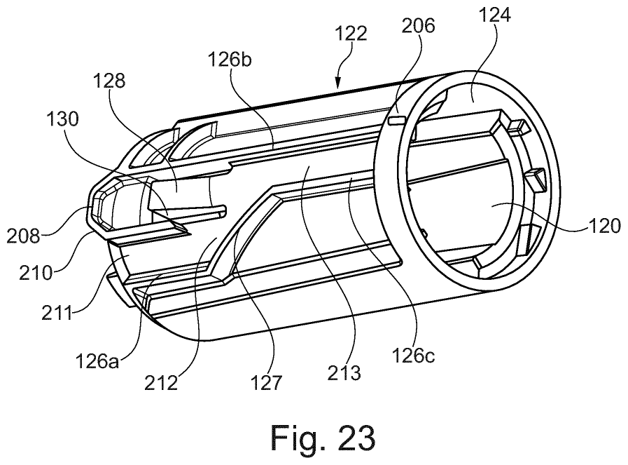

The free ends of the arms 92 of the body 88 are arranged with outwardly directed protrusions 118 that are intended to cooperate with inner surfaces 120 of a generally tubular rotator 122 that is arranged outside and coaxial with the body 88 of the actuator 84. The inner surface 120 of the rotator 122 is arranged with longitudinally extending grooves 124, FIG. 23, the function of which will be described below. The outer surface of the rotator 122 is arranged with a plurality of guide ledges or ribs 126a-c and 127 that define one or more guide tracks 211-213, where some of the guide ledges are extending in the longitudinal direction 126a, 126b, & 126c and some are inclined as represented by section 127 in relation to the longitudinal direction as will be explained. Adjacent one longitudinal guide rib 126b, a proximally directed tongue 128 is arranged, which tongue 128 is flexible in the generally radial direction. The free end of the tongue 128 is arranged with an outwardly directed, wedge-shaped, protrusion 130.

To activate the medicament device 10 of the present disclosure the needle cover 62 must be pressed against an injection site causing it to retract into the housing 12 where the protrusions 76 will move towards the proximal end of rotator 122 such that the protrusion 76 will enter track 211. To prevent misalignment or jamming of the sliding motion of the needle cover 62 relative to the housing 12, the edge 208 and angle edge 210 are provided to guide the protrusion 76 into track 211. The needle cover 62 will move first along the track 211 following guide ledge 126a. Initially, the user must only overcome the biasing force of compression spring 78. However, as the needle cover 62 is pushed further into the housing 12 the protrusion 76 will encounter the angled change in the wall of the guide ledge 127, which could consist of two more angled surfaces, that defines guide track 215, prior to entering guide track 215. As the needle cover 62 is pushed along the angled changes the spring 78 is further compressed. This requires the user to exert more force than would be needed than simply further compressing the spring 78 along a straight linear track. Engagement with the angled guide ledges causes the rotator 122 to start to rotate clockwise relative to the needle cover 62, the body 88 and the housing 12. When a multi-angled surface 127 is used, as the protrusion travels past a first angled guide ledge the user will notice a drop in the required exertion force as the needle cover 62 moves past the point of no return. Further axial movement in the distal direction of needle cover 62 along an adjoining second angled guide ledge will cause further rotation of the rotator 122. Once the rotator 122 has completed its rotation, the protrusion 76 aligns with and enters guide track 215. At this point, the exertion force drops off further and then continues to increase as the compression of spring 78 is further compressed until the needle cover 62 achieves the retracted position as indicated in FIG. 21.

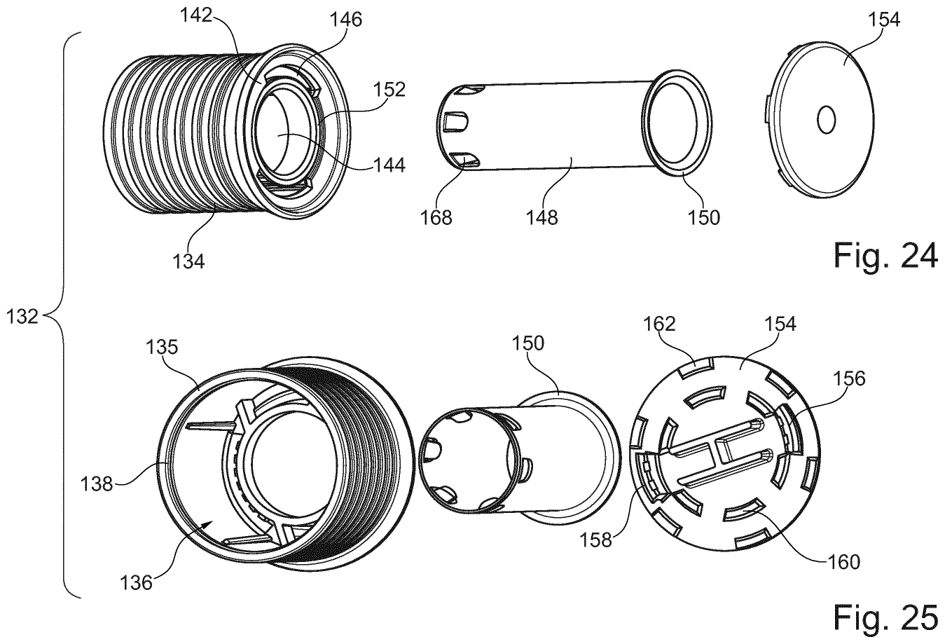

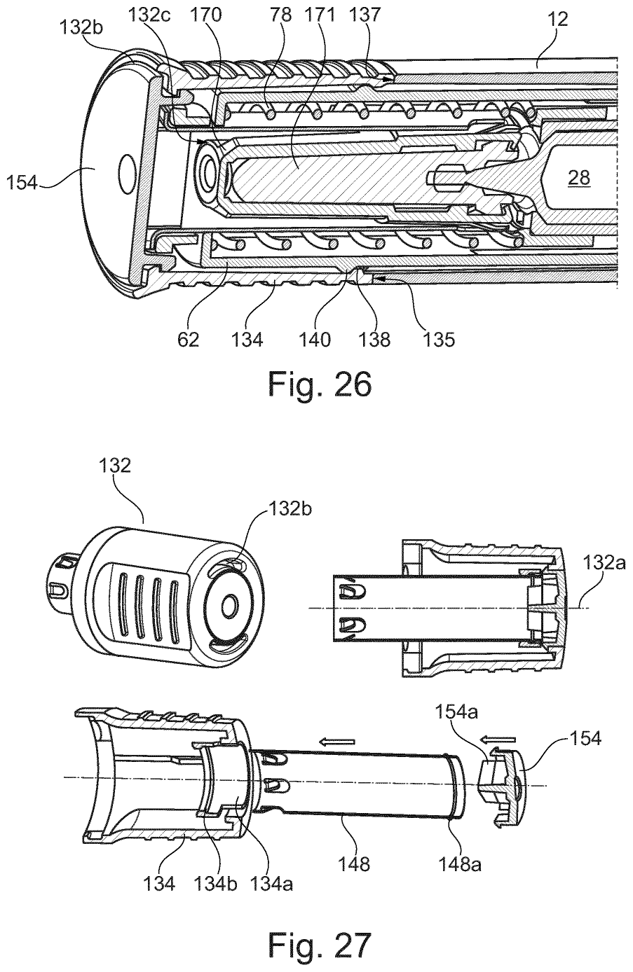

The medicament delivery device 10 is further arranged with a safety cap 132, FIGS. 1 and 24-27, comprising a generally tubular body 134 having a distal passage 136. In order to provide a good fit between the safety cap 132 and the housing 12 of the medicament delivery device 10, the inner surface of the body 134 of the safety cap 132 may be arranged with a circumferential ledge 138, which ledge 138 is arranged to interact with the protrusions 140 on the outer surface of the body 64 of the medicament delivery member guard 62 as seen in FIG. 26. The body 134 of the safety cap 132 is further arranged with a distally directed end surface 135 that acts as an abutment surface against a proximally directed end surface, e.g., rim 137 of the housing 12, which rim 137 also acts as an abutment surface such that the surfaces 135, 137 provide a specific position of the protection safety cap 132 when mounted onto the medicament delivery device 10.

The body 134 of the safety cap 132 is arranged with a proximal end wall 142, which end wall 142 is arranged with a central circular passage 144. Radially outside the central passage 144 are two oppositely positioned arc-shaped openings 146. Alternatively, only one opening of a suitable shape can be provided (not shown here). A generally tubular medicament delivery member shield remover 148 is to be positioned in the central passage 144 of the end wall 142, wherein the medicament delivery member shield remover 148 will extend into the body 134 of the safety cap 132. The proximal end of the medicament delivery member shield remover 148 is arranged with an outwardly extending ledge 150, which ledge or flange 150 is arranged to be seated in a seat or a recess 152 in the end wall 142 of the body 134. The medicament delivery member shield remover 148 is held fixed in place in this position by an end lid 154. The end lid 154 is arranged with a couple of distally directed arc-shaped arms 156 as illustrated in FIG. 25, provided with radially outwardly directed ledges 158, wherein the arms 156 are designed to fit into the arc-shaped openings 146 of the body 134 and the ledges 158 will snap around edges of the arc-shaped openings 146, locking the end lid 154 to the body 134 of the safety cap 132. Alternatively, only one distally directed arm of a suitable shape or an arm comprising a couple of flexible fingers (now shown) can be arranged. The end lid 154 is further arranged with one or a number of distally directed protrusions or ledges 160 which are to be in contact with the ledge 150 of the medicament delivery member shield remover 148, holding it in place in the recess 152.

The distal end of the medicament delivery member shield remover 148 is arranged with generally proximally and inwardly inclined tongues 168 that are designed to be in contact with and engage a medicament delivery member shield such as a rigid needle shield 170 (FIG. 1) or a flexible needle shield 172 or a combination of both, covering the medicament delivery member 58, such as the injection needle. The protective safety cap 132 has a facilitated assembly procedure in that the medicament delivery member shield remover 148 is simply entered into the central passage 144 of the end wall 142 of the body 134 from the proximal side until the ledge 150 is seated in the recess 152 in the end wall 142. Then the end lid 154 is simply pushed onto the proximal end of the body 134 such that the arms 156 fit into the openings and the ledges 158 snap around the edges of the openings 146 of the body 134. A very simple, fast and effective assembly way of the protective safety cap is obtained by the solution. The cap assembly shown in FIG. 26 also has an opening or gap 132b on the proximal end of the body 134 that provides an air passage through the safety cap 132 and prevents possible suffocation should a child for example put the safety cap 132 in the mouth. This is illustrated by the directional arrow 132c

Yet another possible design of the safety cap assembly 132 is illustrated in FIG. 27. Although this design is also as a three-component configuration, this design has a distally facing circular shield remover connector 132a that has an extended groove 132b on an inner portion that is configured to allow for easy engagement with a corresponding outer protrusion 148a on the proximal end of the delivery member shield remover 148. The outer protrusion 148a is sized to form a secure snap fit with inner extended grooved to axially fix the remover shield relative to the inside of the proximal end of body 134 but allows rotation of the remover relative to the 134a. The distally extending ribs 154a on the inside surface of end lid 154 prevent or greatly reduce canting, tilting or otherwise moving out of alignment of the shield remover with and relative to the longitudinal axis 132a of the cap assembly 132. As indicated by the directional arrows in FIG. 12, the shield remover is attached to the body 134 first followed by insertion and connection of the end lid 154. This simplifies the assembly of safety cap 132, yet ensures that the shield remover is securely axially fix to body 134. The cap assembly shown in FIG. 27 also has openings 132b on the proximal end of the body 134 that provides an air passage through the safety cap 132 and prevents possible suffocation should a child for example put the safety cap 132 in the mouth.

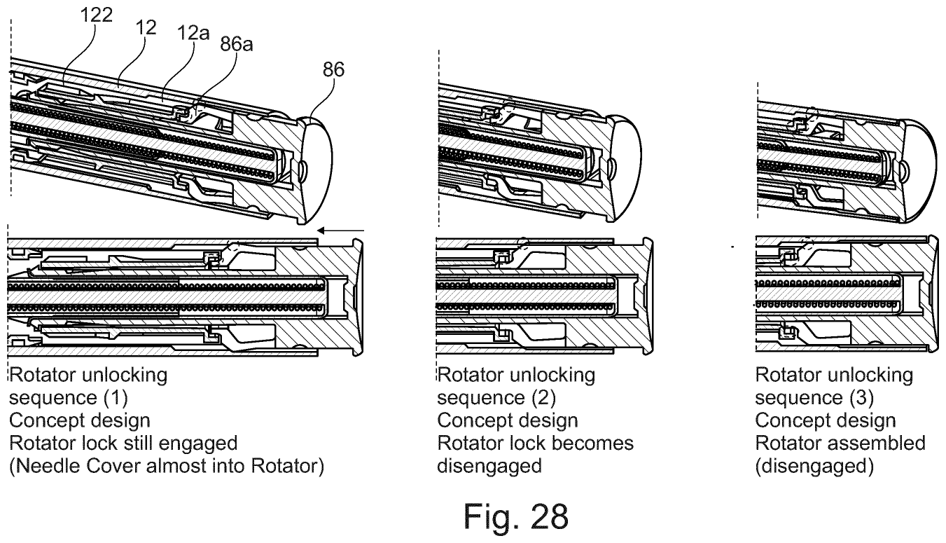

FIG. 28 illustrates one possible embodiment of a rotator transport locking feature 86a that can be used during device assembly to prevent premature or unwanted rotator rotation, which as explained above can cause misalignment of the distal ends of the needle guard 62 with the guide tracks of the rotator. Preferably, the inside of the housing 12 comprises deep grooves 12a at the distal end that becomes shallower as the grooves extend towards the proximal end of the housing. The varying depth of the groove 12a allows the power pack assembly 82 of the injector, which includes the rotator component 122, to be fed further (e.g., 3-4 times longer distance than previously possible) into the housing until the rotation lock 86a on rear cap 86 disengages and the rotator 122 can rotate freely. The needle cover protrusion is then very close to the axial position of the guide track opening on the rotator 122 when the rotation lock 86a is disengaged. The rotator unlocking sequence is shown in FIG. 28.

The medicament delivery device according to the drawings is intended to function as follows. The medicament delivery device is delivered to a user with the safety cap 132 attached to the proximal end of the medicament delivery device. The medicament delivery member guard 62 is in an extended position in relation to the housing 12 such that when the abutment surface 135 of the safety cap 132 is in contact with the abutment surface 137 of the housing, the circumferential ledge 138 is distally of, and in contact with, the protrusions 140 of the medicament delivery member guard 62 as seen in FIG. 26. This provides a very secure fit, reducing the risk for premature release of the safety cap 132.

The medicament delivery device 10 is generally activated by the needle cover 62 being pushed into the housing 12 when the radial flange 68 is pressed against a dose delivery, e.g., injection, site, as will be described. In certain circumstances, activation can be unintended. For example, this may happen accidentally if the medicament delivery device is dropped against a hard surface such as a floor. Such an accident could cause a risk that the medicament delivery device 10 is activated in that the needle cover 62 may be moved in relation to the housing 12 due to the impact forces, which might trigger the medicament delivery device. This risk is reduced or eliminated in that the needle 62 is held by the engagement with the safety cap 132 by the protrusions 140 interacting with the ledge 138.

Once the safety cap 132 is removed, along with the needle shield, the user can then press the radial flange 68 against the injection site, whereby the needle cover 62 is pushed into the 8 12, causing a penetration by the injection needle 58. The movement of the needle cover 62 will cause the protrusions 76 at the distal end of the needle cover 62 to slide in relation to the rotator 122. As the protrusions 76 move along the guide tracks on the outside surface of the rotator 122, the rotator 122 will rotate in relation to the actuator 84, which in turn causes the outward extending protrusions 118 of the arms 92 of the actuator 84 to be moved in position with the longitudinal grooves 124 on the inner surface 120 of the rotator 122. The arms 92 are thereby free to move radially outwards, whereby the engagement between the inwardly directed protrusions 94 and the recesses 95 of the plunger rod 96 is removed, releasing the plunger rod 96. The plunger rod 96 is then urged in the proximal direction by the force of the compressed drive spring 98. The plunger rod 96 will now act on and move the stopper 60 of the medicament container 28 in the proximal direction, expelling a dose of medicament through the injection needle 58. At the end of the injection sequence, the distal end of the plunger rod 96 will pass the bracket 100 whereby the arms 104 of the bracket 100 are free to move radially inwards, wherein the ledges 106 are moved out of contact with the surfaces 108 of the actuator 84. Because the distal end of the drive spring 98 is in contact with the transversal distal part 102 of the bracket 100 via the disk 112 of the guide rod 110 and since the drive spring 98 has a residual force, the bracket 100 will be forced suddenly in the distal direction until the distal end of the bracket 100 hits an end wall of the actuator 84, causing a tactile and audible signal to the user that the injection sequence is completed and that it is safe to remove the medicament delivery device 10 from the dose delivery site.

The user can now remove the radial flange 68 from the injection site which then allows the needle cover 62 to be pushed in the proximal direction by the medicament delivery guard spring 78 that was initially compressed as a result of the needle cover 62 being push in the distal direction during contact of the radial flange 68 and the injection site. The biasing force in the proximal direction caused by the spring 78 decompressing will cause the needle cover and protrusions 76 to move proximally in the guide track 213 such that they come in contact with and pass the wedge-shaped protrusions 130 of the tongues 128 of the rotator 122. The protrusions 76 will ride up and over protrusion 130 until it stops on the proximal side of tongue 128. Passing of the protrusions 130 by protrusions 76 will result in the tongue 128 acting as a hard stop and preventing the protrusions 76 from moving axially in the distal direction. Thus, the needle cover 62 becomes locked in the extended position, covering the injection needle 58, in turn preventing accidental injuries on the injection needle 58. The medicament delivery device 10 can now be discarded.

It is to be understood that the embodiment described above and shown in the drawings is to be regarded only as a non-limiting example that may be modified in many ways within the scope of the patent claims.

1-38. (canceled)

39. A medicament delivery device having a power pack that includes a rotator having an outer surface that is arranged with guide ledges, at least some of the guide ledges being longitudinal guide ledges extending axially in the longitudinal direction and at least some of the guide ledges being inclined guide ledges inclined relative to the longitudinal direction, the guide ledges defining one or more guide tracks that accept and provide a pathway for one or more protrusions located on a distal end of a delivery member guard and wherein axial travel of the delivery member guard and the one or more protrusions within the guide tracks in a distal direction results in activation of the medicament delivery device.

40. The medicament delivery device of claim 39, wherein the one or more guide tracks have two or more non-linear paths that reduce the force needed to activate the medicament delivery device such that activation occurs when the rotator has completed rotation relative to the housing and actuator, and the one or more guide tracks are again parallel to the longitudinal axis of the medicament delivery device, and wherein a completed rotation of the rotator causes release of a plunger rod from a locking engagement with the free ends of flexible arms arranged on the actuator.

41. The medicament delivery device of claim 39, wherein rotation of the rotator occurs when the one or more protrusions of the delivery member guard moving axially and distally in the one or more guide tracks encounter the one or more of the inclined guide ledges.

42. The medicament delivery device of claim 39, wherein an outer surface of the rotator has a radially outwardly extending protrusion positioned on an end of a flexible finger that forms part of a locking mechanism that is activated once the delivery member guard is removed from an injection site after a dose of medicament is administered by the medicament delivery device.

43. The medicament delivery device of claim 42, wherein another part of the locking mechanism is the one or more protrusions of the delivery member guard, and wherein, as the medicament delivery device is moved away from the injection site, the delivery member guard is forced out of the proximal end of the housing of the medicament delivery device by a biasing element and the one or more protrusions simultaneously move proximally within the guide tracks of the rotator until they ride up and over the radially outwardly extending protrusion and the one or more protrusions of the delivery member guard is/are positioned proximally in relation to the radially outwardly extending protrusion such that attempted movement of the delivery member guard in the distal direction is prohibited by the one or more protrusions of the delivery member guard impacting and being stopped by the radially outwardly extending protrusion.

44. The medicament delivery device of claim 39, wherein the delivery member guard is slidably positioned within the proximal end of the housing of the medicament delivery device and has a radial flange located at a terminal proximal end configured for contacting and engaging with an/the injection site.

45. The medicament delivery device of claim 44, wherein an outer diameter of the radial flange is greater than an outer diameter of a proximal tubular portion of the delivery member guard and less than an outer diameter of the housing and the interior diameter of the safety cap.

46. The medicament delivery device of claim 44, wherein the radial flange has one or more notches around its periphery.

47. The medicament delivery device of claim 46, wherein the notches are configured to accept longitudinal ribs positioned on an inside surface of the safety cap to ensure that the safety cap remains removably attached to the medicament delivery device before its intended use.

48. The medicament delivery device of claim 39, wherein the delivery member guard has two parallel longitudinally extending legs at its a distal end.

49. The medicament delivery device of claim 48, wherein the distal ends of the legs include rib guides longitudinally positioned on the outside surface of each leg to form a channel configured to accept housing ribs on the inside of the housing of the medicament delivery device.

50. The medicament delivery device of claim 39, comprising housing ribs positioned longitudinally on the inside of the housing, which housing ribs project radially outward from the inside surface of the housing and have a projection thickness that cooperates with the rib guides on the distal end of the delivery member guard, and wherein cooperation of the rib guides with the housing ribs prevents the delivery member guard from rotating relative to the housing and provides a guiding path or surface to ensure a smooth and non-binding sliding of the delivery member guard during activation of the medicament delivery device.

51. The medicament delivery device of claim 50, wherein the rib guides have a longitudinal length that is approximately equal to the travel length of the delivery member guard during activation.

52. The medicament delivery device of claim 39, wherein one or more protrusions located on a distal end of a delivery member guard are positioned and offset from the terminal end of the legs of the delivery member guard at a distance such that the activation of the medicament delivery device is either delayed or earlier as the radial flange of the delivery member guard is pressed against the injection site.

53. The medicament delivery device (10) of claim 52, wherein the one or more protrusions located on a distal end of a delivery member guard include a chamfer or bevelled surface, preferably is angled 20 degrees or less, to prevent jamming/binding of the legs of the delivery member guard during activation as the one or more protrusions slide in the guide tracks.

54. The medicament delivery device of claim 39, comprising the safety cap releasably connectable to the proximal end of the medicament delivery device, the safety cap having a generally tubular body and a generally tubular medicament delivery member shield remover for removing a medicament delivery member shield.

55. The medicament delivery device of claim 54, wherein the medicament delivery member shield remover comprises an attachment element.

56. The medicament delivery device of claim 54, wherein the safety cap comprises an end lid attached to the generally tubular body so that the medicament delivery member shield remover is held in place in this position by the end lid being in contact with the attachment element of the medicament delivery member shield remover.

57. The medicament delivery device of claim 56, wherein the attachment elements are for holding the generally tubular medicament delivery member shield remover.

58. The medicament delivery device of claim 56, wherein the end lid is attached to said generally tubular body for holding said medicament delivery member shield remover in a fixed position.

59. The medicament delivery device of claim 39, comprising the housing having a proximal end and containing a pre-filled dual chambered medicament container positioned within a container holder rotatably fixed within the housing, where the medicament container has a needle, a dose of medicament and a sliding stopper.

60. The medicament delivery device of claim 59, wherein the container holder has one or more recesses or pockets that facilitate insertion of the pre-filled dual chambered medicament container and interact with the wall portions of windows in the housing to prevent relative rotation.

61. The medicament delivery device of claim 39, wherein the delivery member guard is slidably positioned within the housing to move from a first position to a second position, and then to a final locked position.

62. The medicament delivery device of claim 61, wherein the biasing element for forcing the delivery member guard out of the proximal end of the housing of the medicament delivery device is located at the proximal end of the delivery member guard, and comprises a spring that is in a compressed state when the delivery member guard is in the second position.

63. The medicament delivery device of claim 61, wherein the plunger rod of the medicament delivery device is operatively connected to the stopper and can only move proximally relative to the delivery member guard when the delivery member guard is in the second position.

64. The medicament delivery device of claim 61, wherein the radial flange of the delivery member guard is positioned outside of the proximal end of the housing of the medicament delivery device when the delivery member guard is in the first position, the second position, and the locked position.

65. The medicament delivery device of claim 61, wherein the radial flange of the delivery member guard has an outer diameter greater than the outer diameter of a proximal end portion of the delivery member guard, but less than the outer diameter of the proximal end of the housing.

66. A transport lock assembly for a powerpack of a medicament delivery device, which transport lock is unlocked at the time of final assembly and comprises a plunger rod, an actuator for holding the plunger rod in a pre-tensioned state, a rotator for releasing the plunger rod, and a locking member configured to interact with the rotator, which locking member is movable, relative to the rotator, from a first state in which the rotator is immobilized, to a second state in which the rotator is free to move, the transport lock assembly being further characterized by a housing having a key member, which housing is configured to receive the powerpack, and wherein assembly of the powerpack with the housing causes the key member to move the locking member from the first state to the second state relative to the rotator.

67. The transport lock assembly according to claim 66, comprising a marker on the rotator and a marker on an outer surface of the housing near an opening in the housing, where the marker on the rotator can be viewed through an opening of the housing and alignment of the two markers during the assembly process confirms the correctness of the assembly.

68. The transport lock assembly according to claim 66, comprising a combination of chamfers and blocking gates on an outer surface of the rotator.

69. A rotator for a medicament delivery device, the rotator comprising:
a tubular body extending from a proximal end to a distal end in an axial direction relative to a longitudinal axis; and
one or more ridges extending from a surface of the tubular body, the one or more ridges defining a track on the surface of the tubular body, the track extending in the axial direction from a distal end of the track to a proximal end of the track, the track comprising one pathway at the distal end of the track and two pathways at the proximal end of the track, wherein the two pathways at the proximal end of the track are separated by at least one of the one or more ridges;
and wherein a first pathway of the two pathways at the proximal end of the track is bounded at the distal end of the first pathway by a portion of the one or more ridges, wherein the portion is angled relative to the longitudinal axis, and wherein a first section of the portion can be is at a different angle relative to the longitudinal axis than a second section of the portion.

70. The rotator according to claim 69, wherein the first section of the portion is angled at a larger angle relative to the longitudinal axis than the second portion.

71. The rotator according to claim 69, wherein the second section is closer to the distal end of the rotator than the first section.

72. The rotator according to claim 69, wherein the first section is attached to the second section.

73. The rotator according to claim 69, wherein the second section of the portion is angled at between 10 and 80 degrees relative to the longitudinal axis, or at between 20 and 70 degrees, or at between 25 and 50 degrees.

74. The rotator according to claim 69, wherein the first section of the portion is angled at between 20 and 75 degrees relative to the longitudinal axis, or at between 30 and 70 degrees, or at between 30 and 60 degrees.

75. The rotator according to claim 69, wherein the first section is angled at between 5 and 45 degrees more relative to the longitudinal axis than the second portion, or at between 5 and 35 degrees, or at between 10 and 30 degrees.

76. A medicament delivery device comprising:
a tubular housing that is open at both the proximal and distal ends;
a plunger rod partially assembled and positioned within a tubular proximal portion of the actuator and initially is in a first biased position, the plunger rod being hollow and having a closed proximal end;
an actuator having an end cap located at the distal end and securely fitted into the distal end opening of the housing, wherein a tubular proximal portion of the actuator has a flexible finger that releasably locks the plunger rod when it is in the first biased position;
a compressed drive spring positioned within the plunger rod that biases the closed proximal end of the plunger rod in a proximal direction; and
a rotator is rotationally positioned around the tubular proximal portion of the actuator such that rotation of the rotator relative to the actuator, which is rotationally and axially fixed to the housing, causes the flexible finger to unlock from the plunger rod.
